(19) United States
(12) Patent Application Publication
(10) Pub. No.: US 2008/0252850 A1
(43) Pub. Date: Oct. 16, 2008

(54) Device and Method for the Contactless Determination of the Direction of Viewing

(75) Inventors: Kai-Uwe Plagwitz, Ilmenau (DE); Peter Husar, Homburg (DE); Steffen Markert, Meiningen (DE); Sebastian Berkes, Ilmenau (DE)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(73) Assignee: ELDITH GMBH, Ilmenau (DE)

(21) Appl. No.: 11/663,384

(22) PCT Filed: Sep. 19, 2005
(86) PCT No.: PCT/DE05/01657

§ 371 Date: Mar. 21, 2007

(30) Foreign Application Priority Data
Sep. 22, 2004 (DE) 10 2004 046 617.3

(51) Int. Cl. A61B 3/113

(52) U.S. Cl. 351/210

(57) ABSTRACT

The invention is directed to a device and a method for the contactless determination of the actual gaze direction of the human eye. They are applied in examinations of eye movements, in psychophysiological examinations of attentiveness to the environment (e.g., cockpit design), in the design and marketing fields, e.g., advertising, and for determining ROIs (regions of interest) in two-dimensional and three-dimensional space.

### CROSS-REFERENCE TO RELATED APPLICATIONS

This application claims priority of International Application No. PCT/DE2005/001657, filed Sep. 19, 2005 and German Application No. 10 2004 046 617.3, filed Sep. 22, 2004, the complete disclosures of which are hereby incorporated by reference.

### BACKGROUND OF THE INVENTION

a) Field of the Invention

The invention is directed to a device and a method for the contactless determination of the actual gaze direction of the human eye. They are applied in examinations of eye movements, in psychophysiological examinations of attentiveness to the environment (e.g., cockpit design), in the design and marketing fields, e.g., advertising, and for determining ROIs (regions of interest) in two-dimensional and three-dimensional space.

b) Description of the Related Art

The prior art discloses various devices and methods by which eye gaze direction and gaze point can be determined in a contactless manner.

Corneal reflection method: In this method, the eye is illuminated by one or more infrared light sources so as not to impair vision. The light sources generate a reflection on the cornea which is detected by a camera and evaluated. The position of the reflection point in relation to anatomical features of the eye and those that can be detected by the camera characterizes the eye gaze direction. However, the variability of the parameters of the human eye requires an individual calibration for every eye under examination.

Purkinje eye tracker: These eye trackers make use of camera-assisted evaluation of the light reflected back at the interfaces of the eye from an illumination device whose light impinges on the eye. These Purkinje images, as they are called, occur as a corneal reflection on the front of the cornea (first Purkinje image), on the back of the cornea (second Purkinje image), on the front of the lens (third Purkinje image) and on the back of the lens (fourth Purkinje image). The brightness of the reflections decreases sharply in order. Established devices based on this principle require extremely elaborate image processing and are very expensive.

Search coil method: A contact lens containing thin wire coils is placed on the eye with these wire coils making contact on the outer side. The head of the subject is situated in orthogonal magnetic fields in a time-division multiplexing arrangement. In accordance with the law of induction, an induced voltage is detected for every spatial position of the contact lens synchronous to the magnetic field pulsing. This method is disadvantageous because of the elaborate measurement technique and the cost of the contact lens which holds only about 3 to 5 measurements. In addition, this is a contact method. The contact of the lens is a subjective annoyance to the subject.

Limbus tracking: In this method, reflection light barrier arrangements are placed close to the eye and are oriented to the limbus (margin between the cornea and the sclera). The optical sensors detect the intensity of the reflected light. A shift in the position of the corneal-scleral junction in relation to the sensors and, therefore, the gaze direction can be determined from the differences in intensity. The disadvantage consists in the weak signal of the measurement arrangement which, in addition, sharply limits the visual field which is unacceptable for ophthalmologic examinations.

EOG derivation: From the perspective of field theory, the eye forms an electric dipole between the cornea and the fundus. Electrodes fitted to the eye detect the projection of a movement of this dipole related to eye movement. Typical electric potential curves are approximately linearly proportional to the amplitude of the eye movement. The disadvantage consists in the strong drift of the electrode voltage which is always present and which, above all, prevents detection of static or gradually changing gaze directions. Further, variability between individuals with respect to the dependency of gaze direction on amplitude requires patient-specific calibration. This problem is compounded in that relatively strong potentials of the surrounding musculature are superimposed on the detected signal as interference.

### OBJECT AND SUMMARY OF THE INVENTION

It is the primary object of the invention to provide a device and a method which makes possible a contactless determination of the gaze vector of the human eye without calibrating for every subject.

According to the invention, this object is met in a device for the contactless determination of eye gaze direction in that two cameras are provided, each of which generates images of the human eye simultaneously from different directions, in that the two cameras are connected to an image processing system, and in that at least the spatial coordinates of the cameras and their distance from the eye are stored in the image processing system.

Further, the object of the invention is met through a method for the contactless determination of eye gaze direction in that the eye of a subject is imaged by at least two cameras from at least two different spatial directions, and in that the gaze direction is determined by means of morphological features of an eye which can be evaluated in the image and the spatial coordinates of the cameras and at least their distance from the eye which are stored in the image processing system. When the geometry of the measurement arrangement is known, the gaze point can be determined from the starting point at the eye and from the determined gaze vector. The head need not be fixated, nor must the system be calibrated—as in conventional eye tracking—by correlating a plurality of gaze points and eye positions. The construction is not positioned immediately in front of the eye, but rather can be situated at a sufficient distance from the eye so as not to impair the required visual field (the visible space at a distance of at least 30 cm). The visual field can be further expanded by the arrangement of optical devices such as mirrors, since the photographic systems can now be arranged outside of the visual field. The principle can be applied wherever a fast determination of the actual gaze direction is necessary without impairing the visual field and the well-being of the subject.

The invention will be described more fully in the following with reference to embodiment examples and drawings.

### BRIEF DESCRIPTION OF THE DRAWINGS

In the drawings:

FIG. 1 shows a basic measuring arrangement of the device;

FIG. 2 is a schematic illustration of the measurement principle; and

FIG. 3 shows another illustration of the measurement principle.

### DESCRIPTION OF THE PREFERRED EMBODIMENTS

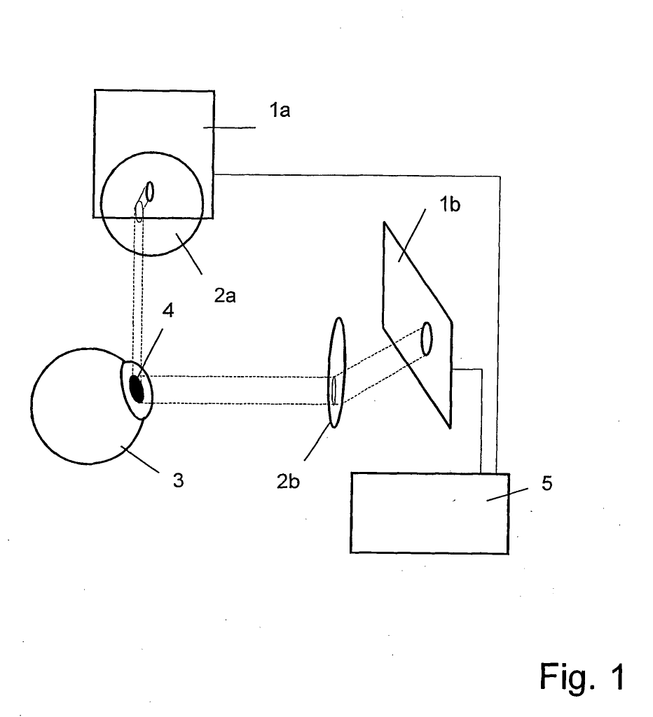

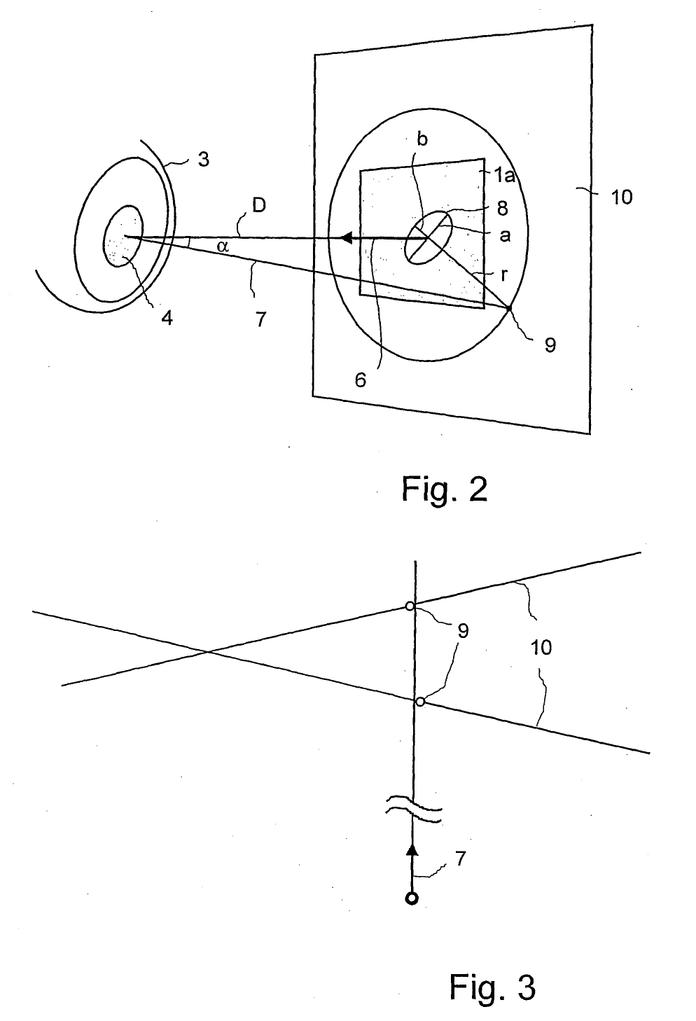

Referring to FIG. 1, the device comprises two cameras, each camera having its essential parts, the receiver surfaces 1a and 1b with their imaging optics 2a and 2b arranged in front. The cameras are located within a spatial reference system (coordinate system). The eye 2 is photographed from at least two spatial directions with simultaneous image recordings. The shape of the pupil 4 and the position on the receiver surfaces 1a and 1b are determined from the images and mathematically described. As can also be seen from FIG. 1, the cameras are connected to an image processing system 5. The surface normal 6 of the respective receiver surface 1a or 1b and the gaze direction vector 7, which is defined as the vector of the tangential surface of the pupil 4, enclose an angle α (FIG. 2). The pupil 4, which is round per se, is imaged as an ellipse 8 through this angle α. The ellipse 8 is characterized by its semimajor axis a and its semiminor axis b. The semimajor axis a corresponds exactly to the radius R of the pupil 4. Further, the distance D (intersection of the axes of the ellipse with the center point of incidence on the pupil 4) is known and is stored in the image processing system 5. The goal is to determine the virtual point 9 from the quantities which are known beforehand and from the measured quantities. The virtual point 9 is the intersection, formed by the straight line of the gaze direction and the projection plane 10, that is given by the receiver surface 1a (FIG. 2). Of course, there is a second virtual point—that of the intersection through the same straight line of the gaze direction and the projection plane—that is formed by receiver surface 1b. The two virtual points need not necessarily coincide. As can be seen from FIG. 3, the determination of the two virtual points can show that they do not lie on a straight line. The gaze direction is then defined by the mean straight line. The simple mathematical equations

$$\begin{matrix}R = tan α * D \\ and & ( 1 ) \\ tan α = \frac{\sqrt{{a}^{2} - {b}^{2}}}{b} \\ give & ( 2 ) \\ r = \frac{\sqrt{{a}^{2} - {b}^{2}}}{b} * D . & ( 3 )\end{matrix}$$

Since the spatial coordinates of the receiver surface 1a are stored in the image processing system 5, the spatial coordinates of the virtual point 9 which characterizes the desired gaze direction can be determined.

An embodiment form of the method will be described in more detail in the following. In the first step, the eye 3 is partially or completely imaged on the image receivers 1a and 1b by the imaging optics 2a and 2b arranged in front. The images are first binarized and the binarization threshold of the gray level distribution is dynamically adapted. The pupil 4 is classified from the binary images and described mathematically approximately as an ellipse. Based on a known algorithm, the two semiaxes a and b, the center point and the angle α are calculated. These parameters depend upon the horizontal and vertical visual angles θ and φ of the eye and upon the dimensions of the pupil and its position in space. The greater semiaxis a is also the diameter of the pupil 4.

Another possibility for realizing the method consists in determining the virtual point by backward projection of characteristic points of the pupil periphery or of points of known position on the pupil from the image to the origin as in trigonometry. It is also possible to arrive at the eye gaze direction by making characteristic diagrams of characteristic curves of b/a−θ−φ and α−θ−φ and determining the intersection of curves of determined parameters.

Instead of the cameras being oriented directly to the eye, the imaging can also be carried out indirectly by means of optical devices which impair the visual field to a much lesser degree.

Investigations of the human eye have shown that the geometric gaze direction vector does not always match the real gaze direction, so that a systematic error can occur. However, the angular deviation is always constant for every subject so that this deviating angle can be included as a correction angle after determining the geometric gaze direction vector. Finally, it should be noted that, within limits, a movement of the head is not critical if it is ensured that 60% of the pupil is still imaged on the receiver surfaces.

While the foregoing description and drawings represent the present invention, it will be obvious to those skilled in the art that various changes may be made therein without departing from the true spirit and scope of the present invention.

### REFERENCE NUMBERS

1a receiver surface
1b receiver surface
2a imaging optics
2b imaging optics
3 eye
4 pupil
5 image processing system
6 surface normal
7 gaze direction vector
8 ellipse
9 virtual point
10 projection plane
a semimajor axis
b semiminor axis
R radius of the pupil
r distance between the center point of the ellipse and the virtual point
D distance
α angle between 6 and 7
φ vertical angle of view
θ horizontal angle of view

What is claimed is:

1-8. (canceled)

9. A device for the contactless determination of eye gaze direction, comprising:
two cameras, each of which generating images of the human eye simultaneously from different directions;
said two cameras being connected to an image processing system; and
at least the spatial coordinates of the cameras and their distance from the center of the pupil of the eye are stored in the image processing system.

10. The device according to claim 9, wherein optical devices are provided in the optical beam path between the eye and cameras for redirecting the images.

11. The device according to claim 9, wherein the correction angle can be stored in the image processing system.

12. A method for the contactless determination of eye gaze direction, comprising the steps of:
imaging the eye of a subject by at least two cameras from at least two different spatial directions; and
determining the gaze direction by morphological features of an eye which can be evaluated in the image and the spatial coordinates of the cameras and their distance from the eye which are stored in the image processing system.

13. The method according to claim 12, wherein gaze is determined based on the mathematical and geometric reconstruction of the position of the characteristic features of the eye in space, wherein these special image features of the eye are described mathematically or geometrically in shape and position and spatial coordinates are assigned to every image point.

14. The method according to claim 12, wherein the features of the eye determining the gaze direction are projected backward in space by means of the imaging characteristics of the arrangement.

15. The method according to claim 12, wherein it can be applied in visible or invisible optical wavelength regions.

16. The method according to claim 12, wherein the geometric gaze direction determined by the image processing system is corrected by a correction angle between the geometric gaze direction and the real gaze direction, which correction angle is determined beforehand by the subject.
